(19) United States
(12) Patent Application Publication
(10) Pub. No.: US 2014/0364365 A1
(43) Pub. Date: Dec. 11, 2014

(54) Mask for Skin Care

(71) Applicant: Far Eastern New Century Corporation, Taipei (TW)

(72) Inventors: Yong-Yi Wu, Zhongli City (TW); Mei-Ting Fan Chiang, Zhongli City (TW); Zij-Jyun Fang, Zhongli City (TW)

(73) Assignee: Far Eastern New Century Corporation, Taipei (TW)

(21) Appl. No.: 14/215,187

(22) Filed: Mar. 17, 2014

(30) Foreign Application Priority Data
Jun. 11, 2013 (TW) 102120750

(51) Int. Cl. A61K 8/73; A61Q 19/00; A61K 8/02; A45D 44/00

(52) U.S. Cl. CPC A61K 8/731; A45D 44/002; A61Q 19/00; A61K 8/0204; A61K 2800/40
USPC 514/9.6; 536/56; 514/17.2; 514/54; 514/25; 514/474; 514/1.1

(57) ABSTRACT

A mask for skin care includes a fiber substrate, wherein the fiber substrate is woven or nonwoven, and includes modified fibers, and the modified fibers are cellulose fibers with at least one hydrophilic functional group grafted on. The grafted ratio of the modified fibers is in the range from 5% to 30%, and the modified fibers can effectively improve the moisture absorption and the moisture-retaining capacity of the mask for skin care.

### RELATED APPLICATIONS

This application claims priority to Taiwanese Application Serial Number 102120750, filed Jun. 11, 2013, which is herein incorporated by reference.

### BACKGROUND

1. Field of Invention

The present invention relates to a mask for skin care. More particularly, the present invention relates to a mask for skin care having high moisture-retaining capacity.

2. Description of Related Art

Recent years, facial mask is so common that daily skin care with the facial mask becomes essential for most female users. There are even facial mask products specifically designed for males. The commercialized facial masks requires the capabilities of moisture retaining, whitening, dark spot correcting, anti-wrinkle, etc. Beside the ingredients of the essence, factor affecting these capabilities is material made of the facial mask. The material as well as the thickness and the texture of the material of the facial mask all affect the moisture absorption, the moisture-retaining capacity, and the fitness of the facial mask. A poor quality nonwoven fabric cannot fit skin tightly, and tends to have the problem of essence dripping, which decreases the absorption capability of the facial mask. Concerning having high concentrated skin care ingredients, if a facial mask product with the same has material with better capability and fitness, it is affirmative to facilitates the absorption of moisture for skin.

The material of facial mask includes nonwoven fabric, jelly, and bio-fermented cellulose, in which the nonwoven fabric is the most common used material mainly because the raw material is easily accessible, less costly, and thus applied extensively. However, the main component of the nonwoven fabric is artificial fiber, such as nylon and polyester. The artificial fiber normally has poorer moisture-retaining capacity than that of natural fiber, e.g., cotton, and leads to problems like worse moisture-retaining capacity and essence dripping when applying. As the result, the effective ingredients of the essence cannot be effectively absorbed by the skin. Further, the poor fitness of the nonwoven mask is inconvenient to users during the application of the mask, which significantly decreases the performance of the facial mask.

In view of the foregoing, bio-cellulose and jelly mask having high moisture-retaining and water holding capacity come into the market recently.

The bio-cellulose mask is fibers obtained by bacterial fermentation, and the fibers are very thin having a diameter between 20 to 100 nm. The nanometer scale fibers are 1/133 of the nonwoven fibers, and smaller than skin, which the sulci cutis is 50 nm in diameter, and thus, the fibers have positive effects on the fitness to skin and the moisture-retaining capacity. However, the cost of bacterial fermentation is high, which is 20 to 30 times higher than that of the traditional nonwoven fabrics, and the high price hesitates the consumers.

The jelly mask is made of many hydrophilic macromolecules as thickening agents, and carboxymethyl cellulose, xanthan gum or the like are used in tradition. Moreover, U.S. Pat. No. 6,379,702 discloses the use of chitosan, U.S. Pat. No. 5,747,022 discloses the use of polyvinyl alcohol, modified acrylate, and modified methacrylate polymer, U.S. Pat. No. 4,640,932 discloses the use of gelatin, starch, cellulose gums, guar gum, alginates, and polyvinyl alcohols, and U.S. Pat. No. 5,139,771 discloses the use of alginic acid, carrageenan, and agar. Mixing the above thickening agents and essence as the contact layer to skin can provide moisture to skin effectively because of the texture of the jelly mask, keep skin supple, equip with water holding capacity, and solve problems of prone to dry and the essence dripping caused by the texture of nonwoven mask. However, most of the current jelly masks are gel or cream type; the users have to dip out the mask spread on face, and after that, wash face with water, which is very inconvenient to the users for the application of the mask.

Given the above, there is a vital need of a mask for skin care that can exclude the above deficiencies in the art. It is expected to provide a mask for skin care solving the inconvenience of jelly mask that need to be washed after applying, having moisture-retaining and water holding capacity as jelly and bio-cellulose mask, and saving more than half of the cost of bio-cellulose mask to meet the users' needs.

### SUMMARY

A mask for skin care is provided. The mask for skin care includes a fiber substrate, wherein the fiber substrate is woven or nonwoven, and includes modified fibers, wherein the modified fibers are cellulose fibers with at least one hydrophilic functional group grafted on, and the grafting ratio of the modified fibers is in the range from 5% to 30%.

In accordance with one embodiment of the present invention, the hydrophilic functional group is a carbonyl group, a carboxyl group, a hydroxyl group, or combination thereof.

In accordance with another embodiment of the present invention, the hydrophilic functional group is a carbonyl group, and the modified fibers are composed of a plurality of segment A and a plurality of segment B, wherein the segment A has the following formula (1), and the segment B has the following formula (2):

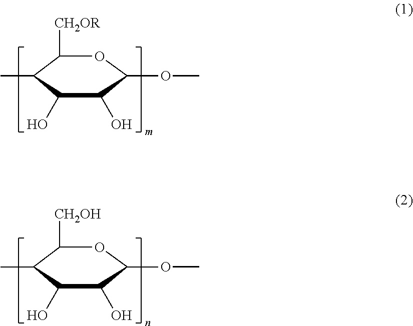

wherein, the segment A and the segment B in the modified fiber has a (m/m+n) ratio in the range from 5% to 30%, and in the formula (1), the R represents CH₂COOH or CH₂COOX, and wherein X is sodium, potassium or ammonium.

In accordance with one embodiment of the present invention, the fiber substrate has a basis weight in the range from 30 to 250 g/m².

In accordance with one embodiment of the present invention, the modified fibers are modified natural fibers, and the natural fibers are cotton fibers, silk cotton fibers, flax fibers, hemp fibers, Manila hemp fibers, pineapple fibers, or combination thereof.

In accordance with another embodiment of the present invention, the mask for skin care further includes at least one effective ingredient, and the effective ingredient is collagen, hyaluronic acid, arbutin, vitamin C, epidermal growth factor (EGF), peptide having biochemical activity, or combination thereof.

In accordance with another embodiment of the present invention, the mask can be applied to facial mask, eye mask, hand mask, or foot mask.

### DETAILED DESCRIPTION

The detailed description provided below is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

A mask for skin care is provided, including a fiber substrate, wherein the fiber substrate is woven or nonwoven, and includes modified fibers, wherein the modified fibers are cellulose fibers with at least one hydrophilic functional group grafted on, and the grafting ratio of the modified fibers is in the range from 5% to 30%.

The modified fibers use chemical modification to introduce the hydrophilic functional group to the cellulose structure and improve the hydrophilicity of the cellulose. Therefore, the mask for skin care made by modified fibers with hydrophilic functional group has good moisture absorption and moisture-retaining capacity.

The hydrophilic functional group described above is a carbonyl group, carboxyl group, a hydroxyl group, or combination thereof. In accordance with one embodiment of the present invention, the hydrophilic functional group is a carbonyl group, and the modified natural fibers are composed of a plurality of segment A and a plurality of segment B, wherein the segment A has the following formula (1), and the segment B has the following formula (2):

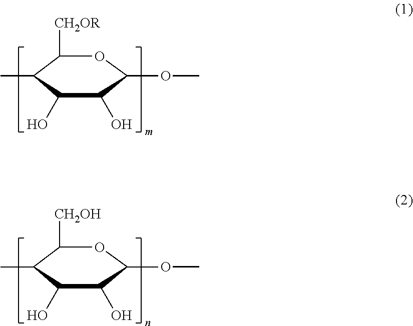

wherein, the segment A and the segment B in the modified fiber has a (m/m+n) ratio in the range from 5% to 30%, and in the formula (1), the R represents CH₂COOH or CH₂COOX, and wherein X is sodium, potassium or ammonium.

In accordance with one embodiment of the present invention, the hydrophilic functional group is —CH₂COONa. The fibers undergo hydrophilic modification by chemical modification, which the —OCH₃ at the C-6 position of the cellulose is modified by —CH₂COONa grafted on, and the hydrophilicity of the cellulose is improved.

In another embodiment, the grafted ratio of the modified fibers is in the range from 10% to 25%. The higher the grafted ratio, the higher the moisture absorption rate of the mask for skin care. However, if the grafted ratio is too high, it will lead the mask for skin care to absorb too much moisture and become too thick affecting the comfort and the fitness to users when applying the mask; if the grafted ratio is too low, the moisture absorption rate will be too low.

In one embodiment, the fiber substrate has a basis weight in the range from 30 to 250 g/m². In another embodiment, the fiber substrate has a basis weight in the range from 60 to 150 g/m². The basis weight of the fiber substrate of mask can affect its mechanical properties. If the basis weight of the fiber substrate of mask is too small, the mechanical properties will become worse; if the basis weight of the fiber substrate is too large, the mask will be too thick and heavy do not be applied easily.

The modified fibers described above are modified natural fibers, and the fibers have cellulose structure, e.g., cotton, silk cotton, flax, hemp, Manila hemp, pineapple fiber, and combination thereof. Artificial fibers having cellulose structure can be applied herein, but, in the view of the base material, the moisture-retaining capacity of artificial fibers is worse than natural fibers.

In accordance with one embodiment of the present invention, the mask for skin care further includes at least one effective ingredient, and the effective ingredient is collagen, hyaluronic acid, arbutin, vitamin C, epidermal growth factor (EGF), peptide having biochemical activity, or combination thereof. However, the effective ingredient is not limited in the above, and can be added as practical needs. For instance, the essence having arbutin can be added to prepare a whitening mask, the essence having anti-wrinkle ingredient can be added to prepare a anti-wrinkle mask, and the essence having hyaluronic acid or collagen can be added to prepare a moisture-retaining mask.

After preparing the above hydrophilic modified fibers, the modified fibers, for example, cotton fibers, are washed with alcohol, drained, and heat dried. Then, opening, carding and needle punching the modified cotton fibers are performed to form a sheet; proceeding by cutting the sheet and soaking in essence, a translucent elastic jelly-like mask is obtained.

The mask for skin care of the present invention can be applied to facial mask, eye mask, hand mask, or foot mask, and the facial mask, eye mask, hand mask, or foot mask with specific efficacy can be prepared as users' needs.

The detailed description provided below is intended as a description of the present examples and is not intended to represent the only forms in which the present example may be constructed or utilized. Therefore, the spirit and scope of the appended claims should not be limited to the description of the embodiments contained herein.

### Chemical

The chemicals needed in the embodiments of the present invention are as following:
1. Sodium mono-chloroacetate: Supplier from Aldrich, US; CAS 3926-62-3; Molecular weight is 116.48.
2. Cotton: Normal cotton.
3. Iso-Propanol: Supplier from Aldrich, US; CAS 67-63-0.
4. Sodium hydroxide: CAS 1310-73-2; Molecular weight is 39.99.
5. Ethanol: CAS 64-17-5; Molecular weight is 46.06.

### Embodiment 1-4: Preparing Modified Fibers

The modification of cotton fibers included following steps:
1. First, the cotton was treated by alkali, which 800 g of cotton was soaked in 10 wt % sodium hydroxide aqueous solution for 2 hours. The crystalline regions in the cellulose structure of the cotton fibers were destroyed after alkali treatment, and swelling phenomenon was occurred to the fibers. Therefore, the moisture absorption capacity of the cotton fibers was improved after alkali treatment.
2. Then, the chemical modification was performed. 30 wt % sodium mono-chloroacetate aqueous solution and 24 wt % iso-propanol aqueous solution was prepared before the reaction. 200 g of alkali treated cotton were soaked in the solution at 60° C. for 1, 3, 5, 7 hours respectively, which were embodiment 1-4. Alkali treated cotton fibers reacted with sodium mono-chloroacetate and underwent carboxymethylation, which improved the moisture absorption capacity of the modified fibers.
3. The reacted fibers were washed by ethanol aqueous solution, heat dried to remove the remaining solution, and the modified natural fibers of embodiment 1-4 were obtained.

### Calculating Grafted Ratio

The grafted ratios of embodiment 1-4 were calculated to confirm the grafted ratios of the modified fibers and the adequate range of the grafted-on in the present invention. The method of calculating the grafted ratio was inferred from the sodium levels of sodium mono-chloroacetate functional group in modified fibers, and the process is described below. Quantified samples (embodiment 1-4) were smashed; sulfuric acid was added to help ashing; samples were placed in 800° C. high temperature furnace ashing for 5 hours to dissolve cellulose, and 2 molecules of sodium mono-chloroacetate reacted with sulfuric acid to produce 1 molecule of sodium sulfate salt. The remaining weights were weighted after cooling, which were the sodium sulfate ash contents of the samples.

Assuming that all sodium mono-chloroacetate reacted at the C-6 position of the cellulose and a pentose was a unit, the definition of the grafted ratio was the ratio of the amount of pentoses having sodium acetate grafted on the C-6 position to total amount of pentoses. Hence, in the formula for defining the grafted ratio, the denominator was the mole of pentoses in the cellulose before grafted, and the numerator was the mole of sodium acetate, which was twice as the mole of sodium sulfate. The mole of pentoses in the cellulose before grafted was obtained by deducting the weight of sodium acetate functional group from the weight of grafted cellulose, and then dividing by the molecular weight of pentose 162. The weight of sodium acetate functional group was twice the mole of the sodium sulfate timing the molecular weight 81. The result was presented in the following Table 1.

The formula of the grafted ratio was as following:

Ash content (%)=W₂ /W₁×100%

W₁: The weight of sample before placing in the furnace, i.e., weight of cellulose after grafted.

W₂: The weight of sample after ashing in 800° C. high temperature furnace, i.e., weight of sodium sulfate.

$$Grafted ratio ( \% ) = \frac{\frac{{W}_{2}}{142} \times 2}{\frac{{W}_{1} - \frac{{W}_{2}}{142} \times 2 \times 81}{162}}$$

162: Molecular weight of pentose.

142: Molecular weight of sodium sulfate.

81: Molecular weight of sodium acetate functional group.

TABLE 1

Embodiment

| | 1 | 2 | 3 | 4 |
|---|---|---|---|---|
| Reacting time (hr) | 1 | 3 | 5 | 7 |
| Ash content (%) | 2.65 | 5.34 | 9.26 | 14.15 |
| Purity of fibers (%) | 97.6 | 97.6 | 97.6 | 97.6 |
| Grafted ratio (%) | 6.08 | 12.64 | 22.99 | 37.4 |

According to Table 1, the grafted ratio of fibers can be controlled by the reacting time of fibers with hydrophilic functional group. However, the higher the grafted ratio does not necessarily be more suitable for preparing mask for skin care. If the grafted ratio is too high, the moisture absorption rate will be too high, and the mask made by the modified fibers will be too thick. Therefore, the adequate grafted ratio can be chosen as practical needs to prepare mask for skin care with different efficacies.

### Embodiment 5-14: Preparing Mask for Skin Care with Different Basis Weights of Fiber Substrate

According to Table 1, embodiment 3 was chosen to prepare mask for skin care with different basis weights of fiber substrate, and the steps were as following:
1. The modified fibers were carded by a carding machine. Embodiment 3 was carded for 1, 1.75, 2.5, 3.5, 5, 6.5, 8, 9, 10, 12 minutes respectively. The purpose of carding was to remove remaining dust and neps, mix fibers thoroughly, and improve the uniformity of fibers.
2. The carded modified fibers were needle punched on both sides to form nonwoven fabrics, the cotton became strong cotton nets, and embodiment 5-14, which were natural fibers masks for skin care with different basis weights, were obtained. Tensile strength test was performed subsequently.

The conditions of the tensile strength test were cutting the dry masks of embodiment 5-14 into 2.5 cm×15 cm, and setting the stretching rate of 0.1 mm/min. The test result was presented in the following Table 2.

TABLE 2

| Embodiment | 5 | 6 | 7 | 8 | 9 | 10 | 11 | 12 | 13 | 14 |
|---|---|---|---|---|---|---|---|---|---|---|
| Take-up time (min) | 1 | 1.75 | 2.5 | 3.5 | 5 | 6.5 | 8 | 9 | 10 | 12 |
| Basis weight (g/m²) | 22.6 | 39.6 | 51.2 | 72.6 | 123.6 | 142.3 | 155.3 | 203.5 | 243.2 | 296.5 |
| Thickness (cm) | 0.12 | 0.19 | 0.26 | 0.41 | 0.72 | 0.86 | 0.95 | 1.28 | 1.56 | 1.89 |
| Lateral tensile | 0.35 | 0.46 | 0.63 | 0.92 | 1.23 | 1.46 | 1.75 | 2.28 | 2.63 | 3.21 |
| strength (kgf) | | | | | | | | | | |
| Longitudinal tensile | 0.62 | 1.35 | 2.23 | 4.65 | 7.69 | 8.22 | 9.39 | 11.05 | 12.63 | 14.66 |
| strength (kgf) | | | | | | | | | | |

According to Table 2, masks for skin care with different basis weights of fiber substrate can be obtained by controlling the take-up time during the carding process, wherein the take-up time is the time that the modified fibers were carded before being taken out of the carding machine. After being made into mask for skin care, if the basis weight is too small, the mechanical strength will become worse; if the basis weight is too large, the fitness to skin will be too poor, and the mask will not be comfortable enough. According to the above test result, the lateral and the longitudinal tensile strength of embodiment 6-13 are preferred for using in mask for skin care. Furthermore, the basis weight in the range from 30 to 250 g/m² is suitable for preparing mask for skin care, wherein the basis weight in the range from 70 to 80 g/m² is preferred.

### Embodiment 15-18: Comparing the Moisture Absorption Capacity of Masks for Skin Care Having Different Grafted Ratio

First, embodiment 1-4 were fabricated into cotton nets having basis weights of about 70-80 g/m², needle punched on both sides, and embodiment 15-18 were obtained. Then, test for moisture absorption capacity was performed, including the steps of:
1. The dry masks of embodiment 15-18 were weighted.
2. The dry masks of embodiment 15-18 were soaked in 100 mL essence for 10 seconds. The ingredients of the essence included pentapeptide, hexapeptide, cucumber extract, mulberry root extract, roselle extract, and hyaluronic acid.
3. The soaked masks were taken out from the essence and weighted.
4. The moisture absorption capacities of the masks were calculated, and the result was showed in Table 3. The method of calculating moisture absorption capacity was as following:

Moisture absorption capacity=[(The weight of soaked mass−The weight of dry mask)÷The weight of dry mask]×100%

TABLE 3

| | | | | | Thickness | |
|---|---|---|---|---|---|---|
| | | | Moisture | | after | |
| | Dry | Wet | absorption | Average | soaking | Average |
| Embodiment | weight (g) | weight (g) | capacity (%) | (%) | (cm) | (cm) |
| 15 | 0.0342 | 0.213 | 522.807 | 570.6434 | 0.25 | 0.21 |
| | 0.0348 | 0.241 | 592.5287 | | 0.21 | |
| | 0.0323 | 0.225 | 596.5944 | | 0.16 | |
| 16 | 0.0312 | 0.35 | 1021.795 | 943.45 | 0.33 | 0.31 |
| | 0.0318 | 0.315 | 890.566 | | 0.31 | |
| | 0.0333 | 0.339 | 918.018 | | 0.28 | |
| 17 | 0.0307 | 0.5822 | 1896.42 | 1970.08 | 0.45 | 0.42 |
| | 0.0318 | 0.6322 | 1988.05 | | 0.42 | |
| | 0.0287 | 0.5814 | 2025.78 | | 0.38 | |
| 18 | 0.0303 | 1.03 | 3299.34 | 3406.149 | 0.68 | 0.69 |
| | 0.0310 | 1.11 | 3480.645 | | 0.72 | |
| | 0.0325 | 1.15 | 3438.462 | | 0.66 | |

According to Table 3, the thickness of embodiment 18 after soaking was 0.69 cm, and the thickness of commercial available facial masks are between 0.1 to 0.4 cm. Thicker structure will be too heavy for face when applying facial mask, and thus embodiment 15-17 are more suitable for making facial mask. Referring to Table 1, embodiment 1-3 have better grafted ratios, and can be inferred that grafted ratio between 5 to 30% has better moisture absorption capacity.

### Comparing the Moisture Absorption Capacity of Masks Made by Different Materials

The test was based on the previous test to evaluate the moisture absorption capacity of commercial available facial masks. Comparison 1, Comparison 2, Comparison 3 and Embodiment 8 were tested, wherein Comparison 1 was nonwoven mask (Whitening mask, Dr. Morita), Comparison 2 was bio-cellulose mask (Bio-cellulose mask, NextDerm), and Comparison 3 was jelly mask (Arbutin+Hyaluronic Acid Brighting Jelly Mask, ANNIE'S WAY).

The testing steps included:
1. Comparison 1, Comparison 2 and Embodiment 8 were cut into 2 cm×2 cm, while 0.3 g of Comparison 3 was spread evenly on a 2 cm×2 cm polyethylene mesh (moisture absorption capacity is less than 0.01%). After drying at 100° C. for 180 minutes, dry masks were obtained.
2. Each dry mask was weighted.
3. Dry mask made by different materials were soaked in 100 mL essence for 10 seconds.
4. The soaked masks were taken out from the essence and weighted.
5. The moisture absorption capacities of the masks were calculated and presented in Table 4.

TABLE 4

| | | | Moisture | |
|---|---|---|---|---|
| | Dry weight | Wet weight | absorption | Average |
| Sample | (g) | (g) | capacity (%) | (%) |
| Comparison 1 | 0.0217 | 0.3251 | 1398.16 | 1489.71 |
| | 0.0175 | 0.2885 | 1548.57 | |
| | 0.0183 | 0.2969 | 1522.40 | |
| Comparison 2 | 0.0103 | 0.0944 | 816.50 | 827.05 |
| | 0.012 | 0.106 | 783.33 | |
| | 0.0107 | 0.105 | 881.31 | |
| Comparison 3 | 0.085 | 0.324 | 281.18 | 330.55 |
| | 0.076 | 0.368 | 384.21 | |
| | 0.08 | 0.341 | 326.25 | |
| Embodiment 8 | 0.0307 | 0.5822 | 1796.42 | 1870.08 |
| | 0.0318 | 0.6322 | 1888.05 | |
| | 0.0287 | 0.5814 | 1925.78 | |

According to Table 4, the moisture absorption capacities of Comparison 1, Comparison 2 and Comparison 3 were 1489.71, 827.05 and 330.55 respectively. The moisture absorption capacity of present embodiment was 1870.08, which was obviously higher than other commercial available facial masks, showing that the mask for skin care of the present invention has high moisture content.

### Method for Evaluating the Moisture Loss Rate

The test was based on the method for testing moisture loss rate to evaluate the moisture loss rate of commercial available facial masks and the mask of the present invention. Comparison 1, Comparison 2, Comparison 3 and Embodiment 8 were tested, wherein Comparison 1 was nonwoven mask (Whitening mask, Dr. Morita), Comparison 2 was bio-cellulose mask (Bio-cellulose mask, NextDerm), and Comparison 3 was jelly mask (Arbutin+Hyaluronic Acid Brighting Jelly Mask, ANNIE'S WAY).

The testing steps included:
1. Comparison 1, Comparison 2 and Embodiment 8 were cut into 2 cm×2 cm, while 0.3 g of Comparison 3 was spread evenly on a 2 cm×2 cm polyethylene mesh. After drying at 100° C. for 180 minutes, dry masks were obtained.
2. Dry masks made by different materials were soaked in 100 mL essence for 10 seconds, while the jelly mask was not.
3. The soaked masks were taken out from the essence and weighted.
4. The soaked masks were placed in a constant temperature and humidity chamber with a temperature of 37° C. and a humidity of 60%, and weighted after 10, 20 and 30 minutes.
5. The moisture loss rates of masks were calculated by following equation:

Moisture loss rate=(The weight of wet mask−The average weight of mask at different time)÷The weight of wet mask×100%

TABLE 5

| | Wet weight | 10 minutes | 20 minutes | 30 minutes |
|---|---|---|---|---|
| Sample | (g) | (g) | (g) | (g) |
| Comparison 1 | 0.3251 | 0.1732 | 0.0796 | 0.0678 |
| | 0.2885 | 0.1819 | 0.0776 | 0.0646 |
| | 0.2969 | 0.1755 | 0.0748 | 0.0677 |
| Average | 0.3035 | 0.1769 | 0.0773 | 0.0667 |
| Moisture loss | 0 | 41.72 | 74.52 | 78.02 |
| rate (%) | | | | |
| Comparison 2 | 0.1419 | 0.096 | 0.0398 | 0.0333 |
| | 0.1452 | 0.0996 | 0.0426 | 0.035 |
| | 0.1762 | 0.1386 | 0.0799 | 0.0575 |
| Average | 0.1544 | 0.1114 | 0.0541 | 0.0419 |
| Moisture loss | 0 | 27.87 | 64.97 | 72.85 |
| rate (%) | | | | |
| Comparison 3 | 0.3256 | 0.2812 | 0.21 | 0.1662 |
| | 0.3369 | 0.2864 | 0.2106 | 0.1798 |
| | 0.3129 | 0.2698 | 0.1965 | 0.165 |
| Average | 0.3251 | 0.2791 | 0.2057 | 0.1703 |
| Moisture loss | 0 | 14.15 | 36.73 | 47.61 |
| rate (%) | | | | |
| Embodiment 8 | 0.835 | 0.8468 | 0.7979 | 0.7654 |
| | 0.8988 | 0.8723 | 0.8261 | 0.8459 |
| | 0.9081 | 0.8901 | 0.9046 | 0.8846 |
| Average | 0.8806 | 0.8697 | 0.8429 | 0.8320 |
| Moisture loss | 0 | 1.24 | 4.29 | 5.53 |
| rate (%) | | | | |

According to Table 5, the moisture loss rate of the present invention was obviously lower than others, showing that the mask for skin care of the present invention had high moisture-retaining capacity.

Given the above, the mask for skin care of the present invention adopt the method of chemical modification to graft hydrophilic functional groups on cellulose structure of fibers, and uses the fibers to make mask for skin care with various functions. The mask for skin care of the present invention can solve the inconvenience of jelly mask that need to be washed after applying, has moisture-retaining and water holding capacity of jelly and bio-cellulose mask, and save half of the cost of bio-cellulose mask.

Although embodiments of the present disclosure and their advantages have been described in detail, they are not used to limit the present disclosure. It should be understood that various changes, substitutions and alterations could be made herein without departing from the spirit and scope of the present disclosure. Therefore, the protecting scope of the present disclosure should be defined as the following claims.

What is claimed is:

1. A mask for skin care comprising:
a fiber substrate, wherein the fiber substrate is woven or nonwoven, and comprises modified fibers, wherein the modified fibers are cellulose fibers with at least one hydrophilic functional group grafted thereon, and have a grafted ratio in the range from 5% to 30%.

2. The mask of claim 1, wherein the grafted ratio of the modified fibers is in the range from 10% to 25%.

3. The mask of claim 1, wherein the fiber substrate has a basis weight in the range from 30 to 250 g/m².

4. The mask of claim 3, wherein the fiber substrate has a basis weight in the range from 60 to 150 g/m².

5. The mask of claim 1, wherein the hydrophilic functional group is a carbonyl group, a carboxyl group, a hydroxyl group, or combination thereof.

6. The mask of claim 5, wherein the hydrophilic functional group is a carbonyl groups, and the modified fibers are composed of a plurality of segment A and a plurality of segment B, wherein the segment A has the following formula (1), and the segment B has the following formula (2): 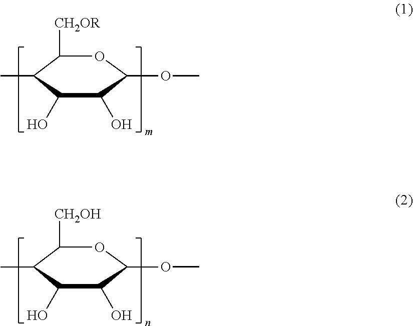
wherein, the segment A and the segment B in the modified fiber has a (m/m+n) ratio in the range from 5% to 30%, and in the formula (1), the R represents CH₂COOH or CH₂COOX, and wherein X is sodium, potassium or ammonium.

7. The mask of claim 1, wherein the modified fibers are modified natural fibers, and the natural fibers are cotton fibers, silk cotton fibers, flax fibers, hemp fibers, Manila hemp fibers, pineapple fibers, or combination thereof.

8. The mask of claim 1, further comprising at least one effective ingredient, and the effective ingredient is collagen, hyaluronic acid, arbutin, vitamin C, epidermal growth factor (EGF), peptide having biochemical activity, or combination thereof.

9. The mask of claim 1, wherein the mask can be applied to facial mask, eye mask, hand mask, or foot mask.
